(19) United States
(12) Patent Application Publication
(10) Pub. No.: US 2016/0143970 A1
(43) Pub. Date: May 26, 2016

(54) NOVEL FORMULATIONS OF BOTANICAL EXTRACTS FOR CANCER THERAPY

(71) Applicant: GENYOUS BIOMED INTERNATIONAL, Redwood Shores, CA (US)

(72) Inventors: James DAO, Henderson, NV (US); Jeffery DAO, San Mateo, CA (US); Thomas DAO, San Francisco, CA (US); David KWOK, Vancouver (CA)

(21) Appl. No.: 14/392,116

(22) PCT Filed: Jun. 3, 2014
(86) PCT No.: PCT/US14/40771

§ 371 Date: Dec. 7, 2015

Related U.S. Application Data
(60) Provisional application No. 61/830,632, filed on Jun. 3, 2013.

(51) Int. Cl. A61K 36/074; A61K 36/537; A61K 45/06; A61K 36/539

(52) U.S. Cl. CPC A61K 36/074; A61K 36/539; A61K 36/537; A61K 45/06

(57) ABSTRACT

Novel formulations are disclosed of a therapeutically effrective composition comprising two or more of an extract of Ganoderma lucidum, an extract of Salvia miltiorrhiza, an extract of Scutellaria barbata, and an extract of Scutellaria baicalensis wherein each extract comprises about 1 to about 90 percent by weight. Extracts are made in aqueous solvents, alcohol solvents and non-alcoholic organic solvents. Formulations comprising one or more emulsifiers demonstrating higher bioavailablity and maximum tolerated dose are provided.

### CROSS-REFERENCE TO RELATED APPLICATIONS

This application claims priority of U.S. Provisional Patent Application Ser. No. 61/830,632, filed Jun. 3, 2013 and titled “NOVEL FORMULATIONS OF BOTANICAL EXTRACTS FOR CANCER THERAPY,” the contents of which are incorporated herein in their entirety by reference.

### TECHNICAL FIELD OF THE INVENTION

The present invention relates to novel and improved compositions of anticancer drugs. This invention relates generally to the field of novel formulations of botanical extracts for ameliorating disease states. Specifically, the invention provides compositions and methods of using botanical extracts in prevention and therapy of diseases including cancer. More specifically, the invention relates to improved formulations comprising therapeutically effective combinations of botanical extracts.

### BACKGROUND OF THE INVENTION

A wide variety of anticancer agents have been developed till date for treatment of various types of cancers in mammals. Novel multifunctional mutitargeted agents directed at cancer, inflammatory and immunological conditions have been derived from botanical extracts.

U.S. Pat. No. 8,173,177 and US Patent App Pub. No. 2005/0214394, both by Dao et al., disclose formulations comprising two or more of an extract of Ganoderma lucidum, an extract of Salvia miltiorrhiza and an extract of Scutellaria barbata that act synergistically in inhibiting cancer. US Patent App Pub. No. 20110117121 (Dao et al.) discloses compositions comprising extracts of the same botanicals for the treatment of pain and related symptoms. The botanical compositions possess therapeutically significant properties and are effective as compositions of the named ingredients and in combination with other botanical extracts and/or chemotherapeutic agents and other pharmacological and chemical entities. These multifunctional multitargeted (MFMT) agents possess multiple functions of therapeutic value and act upon multiple biological targets.

The formulations comprising two or more of an extract of Ganoderma lucidum, an extract of Salvia miltiorrhiza and an extract of Scutellaria barbata are known to have several beneficial effects such as anti-proliferative, antioxidant, immune-boosting, anti-inflammatory, cell cycle modulatory, anti-viral and other activities, while having low toxicity and side effects typically associated with.

Therefore, there is a need for formulations that allow more efficient formulations that allow delivery of increased amounts of these extracts.

### SUMMARY OF THE INVENTION

The present invention relates to compositions and methods of preparation of oral formulations with enhanced bioavailability of botanical extracts comprising synergistically active amounts of extracts of Ganoderma lucidum, Salvia miltiorrhiza, Scutellaria baicalensis and Scutellaria barbata. In some embodiments the extracts are organic extracts. In a preferred embodiment, the extract is an ester (ethyl acetate) extract.

The invention discloses an anti-proliferative formulation comprising: two or more of an extract of Ganoderma lucidum, an extract of Salvia miltiorrhiza, an extract of Scutellaria barbata, and an extract of Scutellaria baicalensis wherein each extract comprises about 1 to about 90 percent by weight; and at least one emulsifying agent. In some embodiments the emulsifying agent is selected from Cremophor EL, oleic acid and labrasol.

The formulation may further comprising an antioxidant. In some embodiments the antioxidant is selected from ascorbic acid and alpha tocopherol.

The formulation may further comprising a diluent. In some embodiments the diluent is soya oil.

In some embodiments the extracts are made in a non-alcoholic organic solvent. In some embodiments the non-alcoholic organic solvent is an ester. In preferred embodiments, the ester is ethyl acetate.

The formulation disclosed exhibits increased bioavailability compared to a formulation without an emulsifying agent and exhibits increased maximum tolerated dose (MTD) compared to a formulation without an emulsifying agent.

The preferred formulation is disclosed, comprising: an ethyl acetate extract of Ganoderma lucidum, an ethyl acetate extract of Salvia miltiorrhiza, and an ethyl acetate extract of Scutellaria barbata wherein each extract comprises about 1 to about 90 percent by weight; at least one emulsifying agent selected from Cremophor EL, oleic acid and labrasol; an antioxidant selected from ascorbic acid and alpha tocopherol; and a diluent selected from soya oil.

The disclosed formulations may further comprise a chemotherapeutic agent. In some embodiments the chemotherapeutic agent is selected from carboplatin, Navelbine® (vinorelbine), anthracycline (Doxil), lapatinib (GW57016), Herceptin, gemcitabine (Gemzar®), capecitabine (Xeloda®), alimta, cisplatin, 5-fluorouracil, epirubicin, cyclophosphamide, avastin, Velcade®, paclitaxel and docetaxel.

In some embodiments the chemotherapeutic agent is selected from the group consisting of antimetabolites, nucleoside analogs, platinum-based agents, alkylating agents, tyrosine kinase inhibitors, anthracycline antibiotics, vinca alkloids, proteasome inhibitors, macrolides, and topoisomerase inhibitors.

A method is disclosed for treating a subject suspected of having cancer or a related disease, the method comprising: administering an amount of the formulation according to any of claims 1-15, sufficient for alleviating a symptom of cancer or a related disease.

In some embodiments the method comprises administration of a formulation comprising 0.1 to 100 mg of an active ingredient consisting of two or more of an extract of Ganoderma lucidum, an extract of Salvia miltiorrhiza, an extract of Scutellaria barbata, and an extract of Scutellaria baicalensis.

In some embodiments of the method 1, 1.5, 2. 2.5, 3, 4, or 5 g of the active ingredient is administered daily.

In some embodiments of the method, the formulation is administered in 7, 14 or 28 day cycles of 1, 2, 3, 4, 5, or 6 cycles.

In some embodiments the method further comprises administering a chemotherapeutic agent either in the same formulation or separately administered as part of a therapeutic regimen.

In some embodiments the chemotherapeutic agent is selected from carboplatin, Navelbine® (vinorelbine), anthracycline (Doxil), lapatinib (GW57016), Herceptin®, gemcitabine (Gemzar®), capecitabine (Xeloda®), Alimta®, cisplatin, 5-fluorouracil, epirubicin, cyclophosphamide, Avastin®, Velcade®, paclitaxel and docetaxel.

In some embodiments the chemotherapeutic agent is selected from the group consisting of antimetabolites, nucleoside analogs, platinum-based agents, alkylating agents, tyrosine kinase inhibitors, anthracycline antibiotics, vinca alkloids, proteasome inhibitors, macrolides, and topoisomerase inhibitors.

The present invention and other objects, features, and advantages of the present invention will become further apparent in the following Detailed Description of the Invention and the accompanying Figures and embodiments.

### BRIEF DESCRIPTION OF THE FIGURES

The following drawings form part of the present specification and are included to further demonstrate certain aspects of the present disclosure, the inventions of which can be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIG. 1 shows an extraction platform for botanical extracts of Ganoderma lucidum (#9), Scutellaria barbata (#15), and Salvia miltiorrhiza (#14).

FIG. 2 shows a schematic of the extraction and first stage concentration for the manufacture of the botanical extracts.

FIG. 3 shows photographs of seven different formulations dispersed in water at a dilution of 10× according to Table 3.

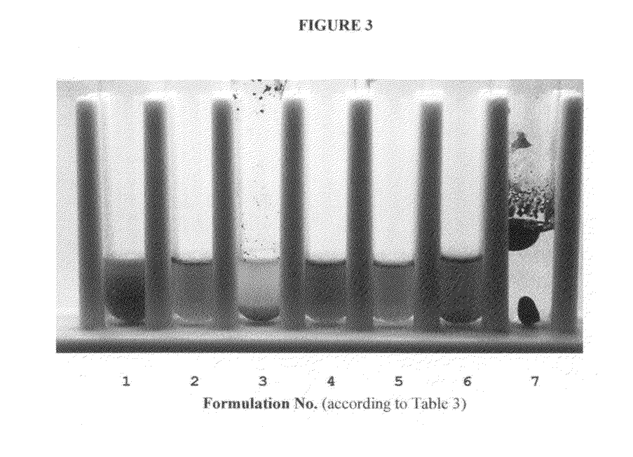

### DETAILED DESCRIPTION OF THE INVENTION

The present invention provides novel methods and botanical compositions for use as multifunctional and multitargeted prophylactics and treatments for human diseases, preferably chronic human diseases. The compositions of the invention have one more of the following properties; anti-inflammatory, anti-oxidant, immune modulating, antiviral, antibacterial, antiproliferative, anticarcinogenic and analgesic. The present invention relates to a novel discovery that botanical extract-based compositions can effectively inhibit numerous response pathways and be substantially less toxic and have less side-effects when administered to an individual.

### DEFINITIONS

The term “plant” as used herein refers to seeds, leaves, stems, flowers, roots, berries, bark, or any other plant parts that are useful for the purposes described. For certain uses, it is preferred that the underground portion of the plant, such as the root and rhizoma, be utilized. The leaves, stems, seeds, flowers, berries, bark, or other plant parts, also have medicinal effects and can be used for preparing tea and other beverages, cream, and in food preparation.

The term “treatment” and “treating” as used herein, for purposes of the specification and claims, includes preventing, inhibiting, curing, or alleviating.

By the term “administering,” it is meant that the compositions are delivered to the host in such a manner that it can achieve the desired purpose. As mentioned the compositions can be administered by an effective route, such as orally, topically, rectally, etc. The compositions can be administered to any host in need of treatment, e.g., vertebrates, such as mammals, including humans, male humans, female humans, primates, pets, such as cats and dogs, livestock, such as cows, horses, birds, chickens, etc.

“Synergism” may be measured by combination index (CI). The combination index method was described by Chou and Talalay. (Chou, T.-C. The median-effect principle and the combination index for quantitation of synergism and antagonism, p. 61-102. In T.-C. Chou and D. C. Rideout (ed.), Synergism and antagonism in chemotherapy. Academic Press, San Diego, Calif. (1991); Chou, T.-C., and P. Talalay. Quantitative analysis of dose-effect relationships: the combined effects of multiple drugs on enzyme inhibitors. Adv. Enzyme Regul. 22:27-55 (1984)). A CI value of 0.90 or less is considered synergistic, with values of 0.85 being moderately synergistic and values below 0.70 being significantly synergistic. CI values of 0.90 to 1.10 are considered to be nearly additive and higher values are antagonistic.

TABLE 1
Synergism/antagonism as a function of CI values

| CI Value | Interpretation |
|---|---|
| >10 | Very strong antagonism |
| 3.3-10 | Strong antagonism |
| 1.45-3.3 | Antagonism |
| 1.2-1.45 | Moderate antagonism |
| 1.1-1.2 | Slight antagonism |
| 0.9-1.1 | Additive |
| 0.85-0.9 | Slight synergism |
| 0.7-0.85 | Moderate synergism |
| 0.3-0.7 | Synergism |
| 0.1-0.3 | Strong synergism |
| <0.1 | Very strong synergism |

It is noted that determination of synergy may be affected by biological variability, dosage, experimental conditions (temperature, pH, oxygen tension, etc.), treatment schedule and combination ratio.

### Botanicals Useful in Preparing Composition According to the Inventions

Botanicals are a valuable resource for the discovery and development of novel, naturally derived agents to treat human disease. Botanical extracts usually comprise multiple molecules and possess multiple functions useful in the treatment and prevention of disease. As a result, botanical extracts can alleviate the harmful side effects of many therapeutic agents used to treat multiple disease targets.

Botanicals have been demonstrated to be a successful source of anticancer compositions. Examples include Gynostemma pentaphyllum extract, Camellia sinensis (green tea) and Crataegus pinnatifida (hawthorn berries) and a method of making the same are the subject of U.S. Pat. Nos. 5,910,308 and 6,168,795. Some drugs, derived from plants that are currently used in cancer therapy were designed to perturb microtubule shortening (depolymerization) or lengthening (polymerization), such as paclitaxel, docetaxel, etoposide, vincristine, vinblastine, and vinorelbine (Compton, D. A., et al., (1999) Science 286:913-914). They share a common mechanism of action of binding to tubulin, the molecule of which microtubules are composed. (Compton, D. A., et al., (1999) Science 286:913-914). At least six plant-derived anticancer agents have received FDA approval (e.g., taxol, vinblastine, vincristine, topotecan, etoposide, teniposide). Other agents are being evaluated in clinical trials (e.g., camptothecin, 9AC, and irinotecan). Botanical extracts for the treatment cancer are described in U. S. Pat. Application Publications 20050214394 A1, 20050208070 A1 and 20050196409 A1.

The present invention provides novel compositions comprising botanical extracts to treat human diseases that are associated with multiple biological pathways in their pathologies. The compositions of the invention are comprised of two or more botanical extracts which work synergistically to modulate multiple biological pathways including but not limited to inflammatory responses, immune responses, oxidative responses, viral and microbial infections, and cell proliferative responses.

(i) Ganoderma lucidum (Reishi): Ganoderma lucidum was praised for its effect of increasing memory and preventing forgetfulness in old age reported in Shen Nong Ben Cao Jing vol. 1 as early as 456-536 AD. Research on mice using orally or topically administered Ganoderma lucidum suggests that Ganoderma lucidum has anti-inflammatory activity. (Stavinoha, W., et al., (1995). Study of the anti-inflammatory efficacy of Ganoderma lucidum. In B.-K. Kim, & Y. S. Kim (Eds.), Recent Advances in Ganoderma lucidum research (pp. 3-7). Seoul Korea: The Pharmaceutical Society of Korea).

Applications of Ganoderma for (1) chemoprophylaxis of cancer in individuals at high risk for developing cancer (2) adjuvant use in the prevention of metastasis or recurrence of cancer (3) palliation of cancer related cachexia and pain and (4) adjunctive use with concurrent chemotherapy to reduce side-effects, maintain leukocyte counts and allow a more optimal dosing of chemo or radio therapeutics has been suggested (Chang, R. (1994) Effective Dose of Ganoderma in Humans; Proceedings of Contributed Symposium 59A, B 5th International Mycological Congress, Vancouver: pp. 117-121). Since studies of human dosage were traditional and empirical, a proper dose range of Ganoderma for therapy was calculated using this data and pharmacokinetic principals. The calculations suggested that a (1) Ganoderma dried fruit body dose of 0.5 to 1 g per day for health maintenance (2) 2 to 5 g per day if there is chronic fatigue, stress, auto immune, or other chronic health problems (3) 5 to 10 g per day for serious illness. (Chang, R. (1993) Limitations and Potential applications of Ganoderma and related fungal polyglycans in clinical ontology; First International Conference on Mushroom Biology and Mushroom products: 96).

While Ganoderma lucidum is preferred, one skilled in the art would recognize that other species of Ganoderma may also be used in the present invention. For example, G. tsugae has been shown to modulate Th1/Th2 and macrophage responses in allergic murine model, and recombinantly expressed fungal immunomodulatory protein, FIP-gts, from G. tsugae inhibited telomerase activity in A549 human lung adenocarcinoma cell line (Lin, J. Y. et al., (2006) Food Chem. Toxicol.; Liao, C. H. et al., (2006) Mo. Carcinog. 45(4):220-9). Examples of other species of Ganoderma include, but are not limited to, G. applanatum, G. mongolicum, G. microsporum, G. subamboinense, G. pfeifferi, G. meredithae, G. oregonense (G. oregonse), G. resinaceum, G. oerstedii, G. ungulatum, G. mirabile, G. tsugae, G. sessile, G. valesiacum, G. fornicatum, G. carnosum, G. australe, and G. boninense.

(ii) Scutellaria barbata (Skullcap): Scutellaria barbata, a traditional Chinese medicine for liver, lung and rectal tumors, has been shown to inhibit mutagenesis, DNA binding and metabolism of aflatoxin B1 (AFB1) and cytochrome P450-linked aminopyrine N-demethylase (Wong B. Y. et al., (1993) Eur. J. Cancer Prev. 2(4):351-6; Wong B. Y. et al., (1992) Mutat. Res. 279(3):209-16). Scutellaria barbata is also capable of enhancing macrophage function in vitro and inhibiting tumor growth in vivo (Wong B. Y. et al., (1996) Cancer Biother. Radiopharm. 11(1):51-6).

This herb contains vitamins C and E as well as calcium, potassium, magnesium, iron, zinc scutellarin, volatile oil, tannin and bitter principles. The scutellarin acts on the central nervous system. Scutellarin, an active ingredient from Scutellaria barbata has been purified by liquid chromatography (Wenzhu Zhang et al., (2003) J. of Liquid Chromatography & Related Technologies 26 (13):2133-40).

(iii) Scutellaria baicalensis: Scutellaria baicalensis has been shown to have anti-proliferative and apoptotic activities against lymphocytic leukemia, lymphoma, and myeloma cell lines and possess anti-cancer activity on human malignant brain tumor cells (Kumagai, T. et al. (2006) Leuk. Res.; Scheck, A. C. et al., (2006) BMC Complement Altern. Med. 6:27).

While Scutellaria barbata and Scutellaria baicalensis are preferred, one skilled in the art would recognize that other species of Scutellaria may also be used in the present invention. For example, Scutellaria radix has been shown to suppress ethanol-induced caspase-11 expression and cell death in N(2)a cells, and Baicalein, a component of Scutellaria radix, leads to suppression of proliferation and induction of apoptosis in human myeloma cells (Kang, K. et al., (2005) Brain Res. Mol. Brain Res. 142(2):139-45; Ma, Z. et al. (2005) Blood 105(8):3312-8). Examples of other species of Scutellaria include, but are not limited to, Scutellaria amabilis, Scutellaria radix, Scutellaria rehderiana, and Scutellaria lateriflora. Preferred combinations are those where the extract from a particular species acts in synergy with extracts from other botanicals in the formulation or with other therapeutic agents in the composition.

(iv) Salvia miltiorrhiza (Dan Shen): There are over 900 species of salvia and many of them have histories of medicinal uses. Dan shen is used in traditional Chinese medicine to promote blood circulation and to remove blood stasis (Bensky D., Gamble A Chinese herbal Medicine Materia Medica 1987 Eastland Press: Seattle. 384). It increases the activity of SOD in platelets, thus providing protection against pulmonary embolism and inhibition of platelet aggregation. (Wang, X. et al., (1996) Zhongguo Zhong Yao Za Zhi 21:558-60). Salvia miltiorrhiza has been shown to lower cholesterol, reduce endothelial damage and to inhibit lipid peroxidation in hypercholesterolemic animals. This inhibition of oxidation of LDL may reduce atherosclerosis (Wu Y. J. et al., (1998) Arteriosclerosis Thromb Vasc Biol 18:481-6). A Salvia miltiorrhiza constituent has been found to inhibit noradrenalin-induced contraction of the aortic strips through reduction in Ca²⁺ mobilization. This vasodilatory activity may explain the traditional use of Salvia miltiorrhiza in hypertension (Nagai M. et al., Biol Pharm Bull (1996) 19:228-32). Salvia miltiorrhiza has been shown to have a markedly superior effect to nitroglycerin, with a more persistent action and better improvement of cardiac function (Bai, Y. R. and Wang, S. Z., (1994) Zhongguo Zhong Xi Yi Jie He Za Zhi 14:24-5, 4).

Salvia miltiorrhiza is also the top ingredient in Dan Shen Compound. Dan Shen Compound comprises four important herbs for the improvement of peripheral circulation and general wellbeing. The actions of Crataegus levigata are enhanced by the Chinese herb Salvia miltiorrhiza (Dan Shen), the Indian herb Coleus forskohlii and Valeriana officinalis. Chinese herbal medicine utilizes Salvia miltiorrhiza for women's irregularities, abdominal pain, insomnia, hives, hepatitis and mastitis.

(v) Hippophae rhamnoides (sea buckthorn): Sea buckthorn seed oil contains a high content of the two essential fatty acids, linoleic acid and α-linolenic acid, which are precursors of other polyunsaturated fatty acids such as arachidonic and eicosapentaenoic acids. The oil from the pulp/peel of seabuckthorn berries is rich in palmitoleic acid and oleic acid (Chen et al., “Chemical composition and characteristics of seabuckthorn fruit and its oil.” Chem. Ind. Forest Prod. (Chinese) 10 (3), 163-175). The increase in the level of α-linolenic acid in plasma lipids showed a clear improving effect on AD symptoms (Yang et al., (2000) J. Nutr Biochem. 11(6):338-340). These effects of α-linolenic acid may have been due to both changes in the eicosanoid composition and other mechanisms independent of eicosanoid synthesis (Kelley (1992) Nutrition, 8 (3), 215-2).

Antioxidant and immunomodulatory properties of seabuckthorn (Hippophae rhamnoides) have been demonstrated using lymphocytes as a model system. (Geetha et al. J Ethnopharmacol 2002 March; 79(3):373-8). The antiulcerogenic effect of a hexane extract from Hippophae rhamnoides has also been demonstrated. (Suleyman H. et al., (2001) Phytother Res 15(7):625-7). Radioprotection by an herbal preparation of Hippophae rhamnoides against whole body lethal irradiation in mice suggests free radical scavenging, acceleration of stem cell proliferation and immunostimulation properties. (Goel H. C. et al., (2002) Phytomedicine 9(1):15-25)

(vi) Camellia sinensis (Green tea): Dried leaves from the Camellia sinensis plant is processed into three types of tea: oolong tea, black tea, and green tea. Green tea extract is a bioflavonoid-rich, potent extract which is used primarily for fighting free radicals. It has a high content of polyphenols, which are a type of bioflavonoids. In making green tea, the tea leaves are stabilized by moist or dry heat which destroys the enzyme polyphenoloxidase and thus, prevents oxidation of polyphenols. These polyphenols are the main biologically active ingredients in green tea. In preferred embodiments, the green tea is Dragon Well tea or Lung Ching tea.

The polyphenols in green tea are catechins, with multiple linked ring-like structures. Polyphenols are a form of bioflavonoids with several phenol groups. They control both taste and biological action. Catechins, a chemical group of polyphenols possessing antioxidant properties (protecting cells from free radical-mediated damage), include epigallocatechin-3 gallate (EGCG), epigallocatechin, and epicatechin-3-gallate. Recently, ECGC has been shown to be an inhibitor of urokinase (Jankun et al., (1997) Nature 387:561), and quinol-oxidase; enzymes that may be crucial for growth of tumor cells. Epigallocatechin-3 gallate (EGCG) also protects against digestive and respiratory infections.

Ganoderma lucidum, Scutellaria barbata, Scutellaria baicalensis, Salvia miltiorrhiza, and Hippophae rhamnoides (seabuckthorn), and Camellia sinensis (green tea) have been used individually for health promoting and therapeutic purposes. Novel tumor inhibiting, immune boosting, inflammation reducing and anti-oxidative properties observed for compositions comprising a combination of two or more extracts of Ganoderma lucidum, Scutellaria barbata, Scutellaria baicalensis, and Salvia miltiorrhiza and, optionally, Hippophae rhamnoides (seabuckthorn) and Camellia sinensis (green tea) and the synergistic effects demonstrated by novel combinations of two or more of these extracts used in the method according to the present invention are a likely result of combinations of one or more of saponins, flavonoids, and polyphenols present in the extracts.

### Compositions

Compositions of the present invention comprise effective amounts of a combination of two or more extracts of Ganoderma lucidum, Scutellaria barbata, Scutellaria baicalensis, Salvia miltiorrhiza, and optionally, Hippophae rhamnoides (sea buckthorn). Preferred compositions are referred to as Aneustat™ or OMN54.

The botanical source materials used for the pharmaceutical manufacturing of OMN54 are cultivated by Shanghai Wah Pao Chinese Herbal Medicine Company Ltd and Jiangsu Tonghui Biologic Technology Co Ltd PRC.

Reishi mushrooms (Ganoderma lucidum) also known as Mannetake (Japan), ling chih or Ling zhi (China) are a fungus from the Polyporacea family. Although they grow wild in many parts of China they are also cultivated in China, Japan, and Korea (1, 2, 6). The parts used in the preparation of OMN54 are the cap and stem of dried fruit body (sporophore). Salvia or ‘Red Ginseng” (Salvia miltiorrhiza Bunge) also known as Dan Shen, is a root from the Family Labiatae. It is grown throughout China. The parts used for OMN54 are the root and rhizome (3, 8). Scute Barbata or Herba Scutellariae Barbatae (Scutellaria barbata D Don) is also known as Ban Zhi Lian is a member of the family Labiatae. It is grown throughout southeastern China and the parts used for OMN54 are the stems and leaves (4, 10).

In one aspect of the invention, the composition comprises equal amounts of extracts of Ganoderma lucidum, Scutellaria barbata, and Salvia miltiorrhiza. The dosage of the composition can be readily determined by one of skill in the art based on the effective concentrations of compositions shown to display the various properties described in this application.

In an embodiment, the composition comprises equal amount of extracts of Ganoderma lucidum, Scutellaria baicalensis, and Salvia miltiorrhiza. The dosage of the composition can be readily determined by one of skill in the art based on the effective concentrations of compositions shown to display the various properties described in this application.

In another embodiment, the composition comprises a 2:1:1:2 ratio of amount of extracts of Ganoderma lucidum, Scutellaria barbata, Scutellaria baicalensis, and Salvia miltiorrhiza, respectively. The dosage of the composition can be readily determined by one of skill in the art based on the effective concentrations of compositions shown to display the various properties described in this application. Compositions comprising different ratios of the individual extracts can similarly be determined. For example, a composition may exhibit anti-inflammatory effects at one concentration or ratios of combinations of extracts and varying degrees of cytotoxic effects at other concentrations or ratios of combinations of extracts. Any ratio of extracts of two or more of Ganoderma lucidum, Scutellaria barbata, Scutellaria baicalensis, and Salvia miltiorrhiza can be used in the compositions of the invention. It is preferred that each extract is present in the composition in equal amounts or at about 1% to about 90% of the total composition. In some embodiments of the invention, any particular extract comprises at least 1%, 1.5%, 2%, 3%, 5%, 7%, 10%, 15%, 25%, 33%, 40%, 50%, 60%, 66%, 75% or 90% of the composition.

In a further embodiment, the composition comprises a combination of Ganoderma lucidum and one or more extracts of Scutellaria barbata and Scutellaria baicalensis. The dosage of the composition can be readily determined by one of skill in the art based on the effective concentrations of compositions shown to display the various properties described in this application.

Combinations of extracts comprising two or more of Ganoderma lucidum, Scutellaria barbata, Scutellaria biacalensis, and Salvia miltiorrhiza are selected for the abilities to, reduce oxidation, reduce inflammation, boost the immune system and inhibit proliferation of cancer cells. The compositions of the present invention comprise botanical compounds that are useful in the reduction of pain.

The compositions of the invention comprise botanical compounds that can be used in the treatment or prevention of diseases associated with an inflammatory response. The compositions of the invention comprise botanical compounds that can be used in the treatment or prevention of diseases associated with oxidative stress. The compositions of the invention comprise botanical compounds that can be used in the treatment or prevention of diseases associated with a perturbed immune response. In some cases the immune response may be hyperactive; for example, autoimmune diseases, and in other cases the immune response may be hypoactive; for example, immunodeficiency diseases.

The compositions of the invention comprise botanical compounds that can be used in the treatment or prevention of diseases associated infectious agents. The compositions may be used in the treatment or prevention of diseases associated with acute or chronic viral infection. The compositions may be used in the treatment or prevention of diseases associated with acute or chronic microbial (bacteria, yeast, fungi) infection.

The compositions of the invention comprise botanical compounds that can be used in the treatment or prevention of diseases associated with abnormal cell proliferation.

In another embodiment, the compositions of the invention comprise botanical compounds that can be used in the treatment of diseases associated with more than one biological pathway. For example, the composition can be used in the treatment of diseases associated with inflammation and cell proliferation, diseases associated with inflammation and immune responses, oxidative stress and cell proliferation or any other combination of biological responses.

The compositions of the invention comprise botanical compounds that can have multiple therapeutic functions in that they may be used to abrogate the effects of a biological pathway that is associated with more than one disease state. For example, compositions of the invention which possess anti-inflammatory activity may be used in the treatment of diseases associated with inflammation such as arthritis, heart disease, stroke, COPD, cancer and pain.

The compositions of the invention include botanical extracts to treat diseases associated with cell proliferation including but not limited to cancer, heart disease, stroke and COPD.

Cancers intended for treatment with the compositions of the invention include, but are not limited to, acinar carcinoma, acinous carcinoma, alveolar adenocarcinoma (also called adenocystic carcinoma, adenomyoepithelioma, cribriform carcinoma and cylindroma), carcinoma adenomatosum, adenocarcinoma, carcinoma of adrenal cortex, alveolar carcinoma, alveolar cell carcinoma (also called bronchiolar carcinoma, alveolar cell tumor and pulmonary adenomatosis), basal cell carcinoma, carcinoma basocellulare (also called basaloma, or basiloma, and hair matrix carcinoma), basaloid carcinoma, basosquamous cell carcinoma, breast carcinoma, bronchioalveolar carcinoma, bronchiolar carcinoma, bronchogenic carcinoma, cerebriform carcinoma, cholangiocellular carcinoma (also called cholangioma and cholangiocarcinoma), chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliale adenoides, carcinoma exulcere, carcinoma fibrosum, gelatiniform carcinoma, gelatinous carcinoma, giant cell carcinoma, gigantocellulare, glandular carcinoma, granulosa cell carcinoma, hair-matrix carcinoma, hematoid carcinoma, hepatocellular carcinoma (also called hepatoma, malignant hepatoma and hepatocarcinoma), Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, lenticular carcinoma, carcinoma lenticulare, lipomatous carcinoma, lymphoepithelial carcinoma, carcinoma mastitoides, carcinoma medullare, medullary carcinoma, carcinoma melanodes, melanotic carcinoma, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, carcinoma nigrum, oat cell carcinoma, carcinoma ossificans, osteoid carcinoma, ovarian carcinoma, papillary carcinoma, periportal carcinoma, preinvasive carcinoma, prostate carcinoma, renal cell carcinoma of kidney (also called adenocarcinoma of kidney and hypemephoroid carcinoma), reserve cell carcinoma, carcinoma sarcomatodes, scheinderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma vilosum.

In a further embodiment, the compositions of the present invention comprise botanical compounds that are useful in compositions to be administered in conjunction with therapeutic agents for the treatment of disease. These compositions exhibit synergistic action with the therapeutic agent. Synergy can be obtained through anti-inflammatory activity, antioxidant activity, immune modulating activity, antiviral activity, antibacterial activity, and/or antiproliferative activity of the compositions of the invention.

In another embodiment, the compositions of the present invention comprise botanical compounds that are useful in compositions to be administered in conjunction with therapeutic agents in the treatment of disease to alleviate the harmful side effects of the therapeutic agent. In this embodiment, the compositions exhibit activities including anti-inflammatory activity, antioxidant activity, immune modulating activity, antiviral activity, antibacterial activity, and/or antiproliferative activity of the compositions of the invention.

### Formulations

The compositions of the present invention can be in any form which is effective including, but not limited to, dry powders, grounds, emulsions, extracts, and other conventional compositions. To extract or concentrate the effective ingredients of the compositions, typically the botanical part is contacted with a suitable solvent, such as water, alcohol, methanol, mixed solvents, or any other solvents. The choice of the solvent can be made routinely, e.g., based on the properties of the active ingredient that is to be extracted or concentrated by the solvent. Preferred active ingredients of the compositions crenulata include, but are not limited to, salidroside, tyrosol, β-sitosterol, gallic acid, pyrogallol, crenulatin, rhodionin, and/or rhodiosin. These ingredients can be extracted in the same step, e.g., using an alcoholic solvent, or they may be extracted individually, each time using a solvent which is especially effective for extracting the particular target ingredient from the plant. In certain embodiments, extraction can be performed by the following process: Milling the selected part, preferably root, to powder. The powder can be soaked in a desired solvent for an amount of time effective to extract the active agents from the compositions. The solution can be filtered and concentrated to produce a paste that contains a high concentration of the constituents extracted by the solvent. In some cases, the paste can be dried to produce a powder extract of the compositions crenulata. The content of active ingredient in the extract can be measured using HPLC, UV and other spectrometry methods.

The compositions of the present invention can be administered in any form by any effective route, including, e.g., oral, parenteral, enteral, intraperitoneal, topical, transdermal (e.g., using any standard patch), ophthalmic, nasally, local, non-oral, such as aerosol, inhalation, subcutaneous, intramuscular, buccal, sublingual, rectal, vaginal, intra-arterial, and intrathecal, etc. It can be administered alone, or in combination with any ingredient(s), active or inactive, including in a medicinal form, or as a food or beverage additive.

In preferred embodiments of the invention, the compositions are administered orally in any suitable form, including, e.g., whole plant, powdered or pulverized plant material, extract, pill, capsule, granule, tablet or a suspension.

The compositions can be combined with any pharmaceutically acceptable carrier. By the phrase, “pharmaceutically acceptable carriers,” it is meant any pharmaceutical carrier, such as the standard carriers described, e.g., Remington's Pharmaceutical Science, 18th Edition, Mack Publishing company, 1990. Examples of suitable carriers are well known in the art and can include, but are not limited to, any of the standard pharmaceutical carriers such as a phosphate buffered saline solutions, phosphate buffered saline containing Polysorb 80, water, emulsions such as oil/water emulsion and various types of wetting agents. Other carriers may also include sterile solutions, tablets, coated tablets pharmaceutical and capsules. Typically such carriers contain excipients such as such as starch, milk, sugar, certain types of clay, gelatin, stearic acid or salts thereof, magnesium or calcium stearate, talc, vegetable fats or oils, gums, glycols. Such carriers can also include flavor and color additives or other ingredients. Compositions comprising such carriers are formulated by well known conventional methods. Generally excipients formulated with the compositions are suitable for oral administration and do not deleteriously react with it, or other active components.

Suitable pharmaceutically acceptable carriers include but are not limited to water, salt solutions, alcohols, gum arabic, vegetable oils, benzyl alcohols, gelatin, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose and the like. Other additives include, e.g., antioxidants and preservatives, coloring, flavoring and diluting agents, emulsifying and suspending agents, such as acacia, agar, alginic acid, sodium alginate, bentonite, carbomer, carrageenan, carboxymethylcellulose, cellulose, cholesterol, gelatin, hydroxyethyl cellulose, hydroxypropyl cellulose, hydroxypropyl methylcellulose, methylcellulose, octoxynol 9, oleyl alcohol, povidone, propylene glycol monostearate, sodium lauryl sulfate, sorbitan esters, stearyl alcohol, tragacanth, xanthan gum, and derivatives thereof, solvents, and miscellaneous ingredients such as microcrystalline cellulose, citric acid, dextrin, dextrose, liquid glucose, lactic acid, lactose, magnesium chloride, potassium metaphosphate, starch, and the like.

The compositions can also be formulated with other active ingredients, such as anti-oxidants, vitamins (A, C, ascorbic acid, B's, such as B1, thiamine, B6, pyridoxine, B complex, biotin, choline, nicotinic acid, pantothenic acid, B12, cyanocobalamin, and/or B2, D, D2, D3, calciferol, E, such as tocopherol, riboflavin, K, K1, K2). Preferred compounds, include, e.g. creatine monohydrate, pyruvate, L-Carnitine, α-lipoic acid, Phytin or Phytic acid, Co Enzyme Q10, NADH, NAD, D-ribose, amino acids such as L-glutamine, Lysine, chrysin; pre-hormones such as 4-androstenedione, 5-androstenedione, 4(or 5-)androstenediol, 19-nor-4 (or 5-)-androstenedione, 19-nor-4 (or 5-)-androstenediol, Beta-ecdysterone, and 5-Methyl-7-Methoxy Isoflavone. Preferred active ingredients include, e.g., pine pollen, fructus lycii, Hippophae rhamnoides, Ligusticum, Acanthopanax, Astragalus, Ephedra, codonopsis, polygola tenuifolia Willd, Lilium, Sparganium, ginseng, panax notogiseng, Garcinia, Guggle, Grape Seed Extract or powder, and/or Ginkgo Biloba.

Other plants and herbs which can be formulated with the compositions of the present invention includes those mentioned in various text and publications, e.g., E. S. Ayensu, Medicinal Plants of West Africa, Reference Publications, Algonac, Mich. (1978); L. Boulos, Medicinal Plants of North Africa, Reference Publications Inc., Algonac, Mich. (1983); and N. C. Shah, (1982) J. Ethnopharm, 6:294-5.

A formulation according to the invention may comprise biologics and chemical entities, in addition to or in the place of, botanical extracts. Examples of biologics that may comprise a composition according to the invention include but are not limited to blood and blood products, cells, tissues and organs, gene therapy vectors, viral and bacterial vaccines, therapeutic products produced through biotechnology such as antibodies, monoclonal antibodies, and the like.

Pharmaceutically active agents that can comprise a composition according to the invention include, but are not limited to antioxidants, anticarcinogens, anti-inflammatory agents, hormones and hormone antagonists, anti-hypertensive agents, anti-inflammatory agents, tranquilizers, cardiotonic agents, antidepressants, corticosteroids, anti-ulcer agents, anti-allergy agents and anti-obesity agents, antibiotics, antibacterial agents, bacterial agents, and other medically useful drugs such as those identified in, e.g., Remington's Pharmaceutical Sciences, 18th Edition, Mack Publishing Company, 1990. Active pharmaceuticals agents can include polysaccharides and other macromolecules such as peptides, proteins, peptidomimetics, cytokines, nucleotides, nucleosides, genetic materials, saccharides, toxoids, serum vaccines or combinations thereof, and pharmaceutically acceptable salts thereof.

A preferred composition of the present invention comprises, about 1%-100%, preferably about 20-70% of the botanical extract and, optionally, a pharmaceutically-acceptable excipient. Another preferred composition of the present invention comprises, about 1%-99%, preferably about 20-70% of botanical extracts, 0.1-99%, preferably 1-10% of one or more pharmaceutically active agents and, optionally, a pharmaceutically-acceptable excipient.

In one embodiment, the drug product is a liquid contained in a size #4 dark brown/black opaque soft gel capsule intended for oral administration. Each capsule contains 100 mg of Aneustat™ (OMN54) and excipients including subtherapeutic levels of oleic acid, Cremophor EL, labrasol, soy bean oil, vitamin E and ascorbic 6-palmitate.

In some embodiments, the composition according to the invention comprises a chemotherapeutic agent either in a single formulation or separately administered as part of a therapeutic regimen. The composition according to the invention can comprise one or more other chemotherapeutic agents including, but not limited to, carboplatin, Navelbine® (vinorelbine), anthracycline (Doxil), lapatinib (GW57016), Herceptin, gemcitabine (Gemzar®), capecitabine (Xeloda®), alimta, cisplatin, 5-fluorouracil, epirubicin, cyclophosphamide, avastin, Velcade®, etc. In some embodiments, the composition according to the invention are co-administered with a chemotherapeutic agent selected from the group consisting of antimetabolites (including nucleoside analogs), platinum-based agents, alkylating agents, tyrosine kinase inhibitors, anthracycline antibiotics, vinca alkloids, proteasome inhibitors, macrolides, and topoisomerase inhibitors.

Suitable anti-proliferative drugs or cytostatic compounds to be used in a composition according to the invention or in combination with a composition according to the invention of the invention such as Aneustat include anti-cancer drugs. Anti-cancer drugs are well known and include: Acivicin®; Aclarubicin®; Acodazole Hydrochloride®; Acronine®; Adozelesin®; Aldesleukin®; Altretamine®; Ambomycin®; Ametantrone Acetate®; Aminoglutethimide®; Amsacrine®; Anastrozole®; Anthramycin®; Asparaginase®; Asperlin®; Azacitidine®; Azetepa®; Azotomycin®; Batimastat®; Benzodepa®; Bicalutamide®; Bisantrene Hydrochloride®; Bisnafide Dimesylate®; Bizelesin®; Bleomycin Sulfate®; Brequinar Sodium®; Bropirimine®; Busulfan®; Cactinomycin®; Calusterone®; Caracemide®; Carbetimer®; Carboplatin®; Carmustine®; Carubicin Hydrochloride®; Carzelesin®; Cedefingol®; Chlorambucil®; Cirolemycin®; Cisplatin®; Cladribine®; Crisnatol Mesylate®; Cyclophosphamide®; Cytarabine®; Dacarbazine®; Dactinomycin®; Daunorubicin Hydrochloride®; Decitabine®; Dexormaplatin®; Dezaguanine®; Dezaguanine Mesylate®; Diaziquone®; Docetaxel®; Doxorubicin®; Doxorubicin Hydrochloride®; Droloxifene®; Droloxifene Citrate®; Dromostanolone Propionate®; Duazomycin®; Edatrexate®; Eflornithine Hydrochloride®; Elsamitrucin®; Enloplatin®; Enpromate®; Epipropidine®; Epirubicin Hydrochloride®; Erbulozole®; Esorubicin Hydrochloride®; Estramustine®; Estramustine Phosphate Sodium®; Etanidazole®; Etoposide®; Etoposide Phosphate®; Etoprine®; Fadrozole Hydrochloride®; Fazarabine®; Fenretinide®; Floxuridine®; Fludarabine Phosphate®; Fluorouracil®; Flurocitabine®; Fosquidone®; Fostriecin Sodium®; Gemcitabine®; Gemcitabine Hydrochloride®; Hydroxyurea®; Idarubicin Hydrochloride®; Ifosfamide®; Ilmofosine®; Interferon Alfa-2a®; Interferon Alfa-2b®; Interferon Alfa-n1®; Interferon Alfa-n3®; Interferon Beta-I a®; Interferon Gamma-I b®; Iproplatin®; Irinotecan Hydrochloride®; Lanreotide Acetate®; Letrozole®; Leuprolide Acetate®; Liarozole Hydrochloride®; Lometrexol Sodium®; Lomustine®; Losoxantrone Hydrochloride®; Masoprocol®; Maytansine®; Mechlorethamine Hydrochloride®; Megestrol Acetate®; Melengestrol Acetate®; Melphalan®; Menogaril®; Mercaptopurine®; Methotrexate®; Methotrexate Sodium®; Metoprine®; Meturedepa®; Mitindomide®; Mitocarcin®; Mitocromin®; Mitogillin®; Mitomalcin®; Mitomycin®; Mitosper®; Mitotane®; Mitoxantrone Hydrochloride®; Mycophenolic Acid®; Nocodazole®; Nogalamycin®; Ormaplatin®; Oxisuran®; Paclitaxel®; Pegaspargase®; Peliomycin®; Pentamustine®; Peplomycin Sulfate®; Perfosfamide®; Pipobroman®; Piposulfan®; Piroxantrone Hydrochloride®; Plicamycin®; Plomestane®; Porfimer Sodium®; Porfiromycin®; Prednimustine®; Procarbazine Hydrochloride®; Puromycin®; Puromycin Hydrochloride®; Pyrazofurin®; Riboprine®; Rogletimide®; Safingol®; Safingol Hydrochloride®; Semustine®; Simtrazene®; Sparfosate Sodium®; Sparsomycin®; Spirogermanium Hydrochloride®; Spiromustine®; Spiroplatin®; Streptonigrin®; Streptozocin®; Sulofenur®; Talisomycin®; Taxol®; Taxotere®; Tecogalan Sodium®; Tegafur®; Teloxantrone Hydrochloride®; Temoporfin®; Teniposide®; Teroxirone®; Testolactone®; Thiamiprine®; Thioguanine®; Thiotepa®; Tiazofurin®; Tirapazamine®; Topotecan Hydrochloride®; Toremifene Citrate®; Trestolone Acetate®; Triciribine Phosphate®; Trimetrexate®; Trimetrexate Glucuronate®; Triptorelin®; Tubulozole Hydrochloride®; Uracil Mustard®; Uredepa®; Vapreotide®; Verteporfin®; Vinblastine Sulfate®; Vincristine Sulfate®; Vindesine®; Vindesine Sulfate®; Vinepidine Sulfate®; Vinglycinate Sulfate®; Vinleurosine Sulfate®; Vinorelbine Tartrate®; Vinrosidine Sulfate®; Vinzolidine Sulfate®; Vorozole®; Zeniplatin®; Zinostatin®; Zorubicin Hydrochloride®.

Other anti-cancer drugs suitable for combination therapy include: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta-lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-I receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anti cancer compound; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; teltelomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

The present invention relates to methods of administering the compositions, e.g., to provide anti-inflammatory effects, to reduce inflammation, to provide antioxidant effects, to protect against oxidation, to provide antiviral effects, to prevent viral infection, to provide anti-bacterial effects, to prevent bacterial infection, to provide antiproliferative effects, to provide anti-cancer effects, to promote DNA repair, to provide anti-radiation effects, to protect against radiation, and other conditions and diseases as mentioned herein.

An effective amount of the compositions are administered to such a host. Effective amounts are such amounts which are useful to achieve the desired effect, preferably a beneficial or therapeutic effect as described above. Such amount can be determined routinely, e.g., by performing a dose-response experiment in which varying doses are administered to cells, tissues, animal models (such as rats or mice in maze-testing, swimming tests, toxicity tests, memory tests as performed by standard psychological testing, etc.) to determine an effective amount in achieving an effect. Amounts are selected based on various factors, including the milieu to which the composition is administered (e.g., a patient with cancer, animal model, tissue culture cells, etc.), the site of the cells to be treated, the age, health, gender, and weight of a patient or animal to be treated, etc. Useful amounts include, 1 milligram-50 milligrams, 10 milligrams-100 grams, 100 milligrams-10 grams, 250 milligrams-2.5 grams, 1 gm, 2 gm, 3 gm, 500 milligrams-1.25 grams. etc., per dosage of different forms of the compositions such as the botanical powder, botanical extract paste or powder, tea and beverages prepared to contain the effective ingredients of the compositions, and injections, depending upon the need of the recipients and the method of preparation.

Suitably the composition is in unit dosage form. Examples of unit dose formulations of the present invention include capsule and tablet formulations, preferably a capsule formulation.

The liquid, pharmaceutically active formulation comprises a pharmaceutically active composition according to the invention in a liquid diluent or carrier. The active ingredient may be dissolved or dispersed in the liquid diluent or carrier, which may be a water miscible or water immiscible medium. Examples of liquid diluents or carriers include the following three classes: (a) Water miscible carriers: Propylene Glycol, Polyethylene Glycol, Water, Solketal, Glycofurol, Dimethylisosorbide, Nonionic surface active agents; (b) Oils and Organic carriers: Fractionated Coconut Oil, Sesame Oil, Soya Bean Oil, Vegetable Oil, Liquid Paraffin, Isopropylmyristate, Triacetin; and (c) Semi-solid carriers: High molecular weight polyethylene glycols, and White soft paraffin.

Formulations with Emulsifying Agents

It has been surprisingly observed that use of one or emulsifying agents increases bioavailability and maximum tolerated dose of the composition comprising extracts of two or more of Ganoderma lucidum, Scutellaria barbata, Scutellaria baicalensis, and Salvia miltiorrhiza.

In some embodiments, one or more emulsifiers or surfactants are included in the formulation. Suitable emulsifiers which can be used include one or more of fatty acids such as oleic acid, polyoxyethylene glycerol esters of fatty acids, such as Tagats; polooxylated castor oil, ethylene glycol esters, such as glycol stearate and distearate; propylene glycol esters, such as propylene glycol myristate; glyceryl esters of fatty acids, such as glyceryl stearates and monostearates; sorbitan esters, such as spans and tweens; polyglyceryl esters, such as polyglyceryl 4-oleate; fatty alcohol ethoxylates, such as Brij type emulsifiers; ethoxylated propoxylated block copolymers, such as poloxamers; polyethylene glycol esters of fatty acids, such as Labrafils, Labrafacs, and Labrasols; cremophores; glycerol monocaprylate/caprate, such as Campmul CM 10; Gelucire, Capryol, Captex, Acconon, transcutol, triacetin, and the like. In some embodiments, the emulsifying agent is selected from Cremophor EL, oleic acid and labrasol. In some embodiments, antioxidants and/or diluents are used in the formulation. In some embodiments, antioxidants are selected from selected from ascorbic acid and alpha tocopherol. In some embodiments, the diluents is soya oil.

Compositions of the present invention comprise effective amounts of a combination of two or more extracts of Ganoderma lucidum, Scutellaria barbata, Scutellaria baicalensis, Salvia miltiorrhiza, and optionally, Hippophae rhamnoides (sea buckthorn) that exhibit synergy in an ant-proliferation or anti-inflammation assay.

In one aspect of the invention, the botanical composition comprises effective amounts of extracts of Ganoderma lucidum, Scutellaria barbata, and Salvia miltiorrhiza. The dosage of the composition can be readily determined by one of skill in the art based on the effective concentrations of compositions shown to display the various properties described herein.

Extracts of the botanicals were preferably made in organic medium, such as alcohol, ester, lipid and the like. In a preferred embodiment the extracts were made in ethyl acetate medium.

Compositions comprising different ratios of the individual extracts can similarly be determined. For example, a composition may exhibit anti-inflammatory effects at one concentration or ratios of combinations of extracts and varying degrees of cytotoxic effects at other concentrations or ratios of combinations of extracts. Any ratio of extracts of two or more of Ganoderma lucidum, Scutellaria barbata, Scutellaria baicalensis, and Salvia miltiorrhiza can be used in the compositions of the invention. It is preferred that each extract is present in the composition in equal amounts or at about 1% to about 90% of the total composition. In some embodiments of the invention, a particular extract comprises at least 1%, 1.5%, 2%, 5%, 10%, 15%, 25%, 33%, 40%, 45%, 47.5%, 48.5%, 49.5%, 50%, 60%, 66%, 75%, 90% or 98% by weight of the composition. In one embodiment the OMN54 comprises about 1-3% Salvia miltiorrhiza, and approximately equal amounts (45-50%) of Scutellaria barbata and Ganoderma lucidum.

According to the invention, some embodiments comprised a botanical composition comprised of the following three extracts:

(i) Ganoderma lucidum at 33-50% w/w. More specifically the Ganoderma lucidum extract is selected from 33%, 35%, 40%, 42%, 44%, 45%, 46%, 46.5%, 47%, 47.5%, 48%, 48.5%, 49%, 49.5% and 50%.

(ii) Scutellaria barbata at 33-50% w/w. More specifically the Scutellaria barbata extract is selected from 33%, 35%, 40%, 42%, 44%, 45%, 46%, 46.5%, 47%, 47.5%, 48%, 48.5%, 49%, 49.5% and 50%.

(iii) Salvia miltiorrhiza at 1-10% w/w. More specifically the Salvia miltiorrhiza extract is selected from 1%, 1.5%, 2%, 2.5%, 3%, 3.5%, 4%, 4.5%, 5%, 5.5%, 6%, 6.5%, 7%, 7.5%, 8%, 8.5%, 9%, 9.5% and 10%.

These botanical compositions are referred to in the Specification as OMN54.

A series of oral formulations of OMN54 were tested to achieve optimal chemical stability, physical properties for manufacturing of finished product and bioavailability.

TABLE 2
Oral formulations of OMN54.

| | | Rate of dispersion of OMN54 at 50 mg/ml | |
|---|---|---|---|
| # | Formulation | in water at a ratio of 1:10 | Clarity |
| 1 | oleic acid:Cremaphor:labrasol | Fast dissolution and forms a homogenous | A |
| | (20:40:40) | mixture | |
| 2 | 60:40, | Fast dissolution time and forms a | B |
| | Labrasol:Tween80 | homogenous mixture | |
| 3 | 40:10, PG:EtOH | Precipitates out of solution | NG |
| 4 | Labrasol | Fast and forms a homogenous dispersion | B |
| 5 | Tween 80 | Slow dissolution time but forms a | B |
| | | homogenous dispersion | |
| 6 | Cremaphor | Very slow dissolution time but forms a | B |
| | | homogenous dispersion | |
| 7 | Genyous formulation | Forms two phases (no dissolution) | B |

Note:
A−: optically clear microemulsion;
B+: less bio-fringent material;
B: some biofringent material,
NG: not good

An expanded list of additional oral formulations of OMN54 was carried out for the following list of formulations:

1) Cremophor EL

2) Cremophor RH40: Tween80/PG

3) Tween80

4) Geloil SC: Tween80/PG

5) Tween80/PG

6) Geloure 44/14: Tween80/PG

In addition to developing an oral formulation for high bioavailability of OMN54 components, a successful oral formulation is required to provide favorable liquid flow characteristics for capsule manufacturing. Therefore to provide a flowable liquid characteristic to the semisolid paste-like OMN54, a mixture of Tween80:propylene glycol (1:1) was selected as a free-flowing liquid to be used in combination with selected semi-solid excipients.

Each of the test formulations, either containing a single excipient or in combination with Tween80/PG, was added to OMN54 at 30%, 40% and 50% w/w. This range of excipient proportions is intended to show an optimal excipient-to-drug ratio for all of the OMN54 components to be adequately dispersed/dissolved following oral administration.

Dispersion/dissolution media included the use of three independent sets of OMN54 formulations dispersed either in water, acidic pH and alkaline pH aqueous solutions to simulate pH transit of the OMN54 dosage from the stomach to the gut at a 1:50 dispersion ratio. Evaluations were also designed to simulate the physiological dispersion volume and performed both at room temperature and at 37° C.

Since OMN54 is comprised of a mixture of non-water soluble chemical components, the following dispersion/dissolution endpoints were used in the evaluation and comparison of the various oral formulations:
the rate of dispersion (presence of micro-emulsion=fine emulsified colloid of solublized but immiscible drug) and/or dissolution (clarity as a measure of solubilization),
the presence and amount of suspended solids (amount of settled solids as a measure of degree of solubilization),
the presence of settled solids upon low speed centrifugation (differentiate micro-emulsion from suspended solids),
dispersed microemulsion/solid suspension at room temperature and at 37° C. (confirmed in vivo),
dispersed microemulsion/solid suspension in water, acidic and alkaline aqueous solutions (confirmed in vivo).

Bergstrom et al. (Bergstrom D. H., Capsules, Soft, Encyclopedia of Pharmaceutical Technology, 2002, 317-327) discloses that gelatin based capsules are compatible with most water miscible liquids like polyethylene glycols, non-ionic surfactants (Tween, Cremaphor) and lipids (oleic acid).

OMN54 was dissolved in each of the following vehicles at a concentration of 50 mg/ml:
20:40:40 oleic acid: cremaphor: labrasol
60:40, Labrasol:Tween 80
Valium formulation (40:10, propylene glycol:ethanol)
labrasol
Tween 80
Cremaphor

The solutions were then dispersed in water and observed for homogeneity.

Solubility Determination:

All the solutions/suspensions were made using the following procedure: To a weighed amount of the drug, sufficient vehicle was added to give the desired concentration. The samples were put on the end over end mechanical rotator at room temperature and rotated overnight. Dissolution was enhanced by sonnicating the solutions.

All the physical mixtures were made by triturating the desired concentration of drug and excipient until it formed a homogenous mixture:

Simulated Gastric Fluid:

USP procedure without addition of pepsin: 2.0 g of sodium chloride was dissolved in 7 ml of Hydrochloric acid. This was then diluted to a total volume of 1000 ml

Dissolution Study:

Each of the formulations was diluted 10×, with water to test the rate of dissolution and the homogeneity of the dispersion. Table 3 summarizes the visual observations for the addition of formulation to water.

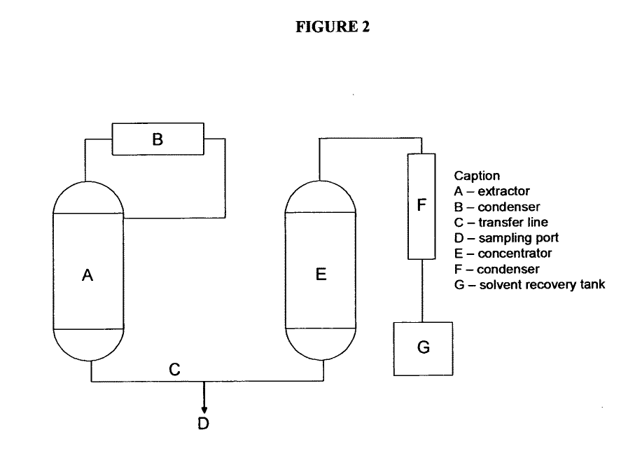

FIG. 2 shows the results of mixing 1 part formulation with 10 parts water.

All of the formulations other than the formulation (#3) produced clear solutions at a 100× dilution. However, the self emulsifying drug delivery system (SEDDS) appeared to be somewhat better than the others.

TABLE 3
Results from Static Dilution Test in water at a drug
concentration of ~50 mg/ml and 10X dilution

| # | Formulation | Rate of dispersion | Clarity |
|---|---|---|---|
| 1 | 20:40:40 oleic acid:Cremaphor:labrasol | Fast dissolution and | A |
| | | forms a homogenous mixture | |
| 2 | 60:40, Labrasol:Tween80 | Fast dissolution time and | B |
| | | forms a homogenous mixture | |
| 3 | 40:10, PG:EtOH | Precipitates out of solution. | NG |
| 4 | Labrasol | Fast and forms a homogenous dispersion | B |
| 5 | Tween 80 | Slow dissolution time but | B |
| | | forms a homogenous dispersion | |
| 6 | Cremaphor | Very slow dissolution time | B |
| | | but forms a homogenous dispersion | |
| 7 | Genyous formulation | Forms two phases (no dissolution) | NG |

A−: optically clear microemulsion;
B+: less bio-fringent material;
B: some bio-fringent material,
NG: not good

### Formulation and Administration

Typical formulations comprise soft gelatin capsules for oral consumption comprising 100 mg of active OMN54 (Aneustat™).

The formulations is administered is cycles of 3, 7, 14, 21, or 28 days. A cycle may be repeated 1, 2, 3, 4, 5, or 6 times in succession with 0, 1, 2, 3, 4, 7, 10, 14, 21 or more days interval in between. A subject receives Aneustat™ (OMN54) on either a once daily (QD) or twice daily (BID) or more frequent schedule through each cycle. The doses are administered at morning or evening, pre- or post-prandial. Repeat doses are administered at 6, 8, 10 or 12 hour intervals.

The daily dose of the formulation administered to a subject can be 100 mg, 200 mg, 500 mg, 700 mg, 1 g, 1.5 g, 2 g, 3 g, 4 g, 5 g or more. Administration is subject to determination of maximum tpolerated dose (MTD) or non-tolerated dose (DLT).

Subjects suitable for administration of the formulation include, but are not limited to, subjects meeting one or more of the following criteria:

1. Histological or cytological evidence of malignancy

2. Male or female, 18 years or older

3. Presence of advanced tumours, i.e., measurable or non-measurable disease as per RECIST criteria, version 1.1, that have recurred or progressed following standard therapy

4. Ability to swallow the oral capsule form of the drug

5. Failed at least one previous therapeutic regimen and either no longer are candidates for standard therapy, have no standard therapy available, or choose not to pursue standard therapy.

### Pharmacokinetics

Pharmacokinetic assessments of the concentrations of Aneustat™ (OMN54) in plasma are performed pre-dose Day 1 and post dose at approximately 0.5, 1, 1.5, 2, 3, 4, and 8 hours.

A selected number of chemical markers for Aneustat (OMN54) have been identified as ganoderic acid A, tanshinone IIA, scutellarein, and apigenin. An electrospray LC/MS/MS assay has been established for the quantitation of these chemical markers in human plasma for the assessment of oral bioavailability and plasma pharmacokinetics. Since a significant portion of these chemical markers in plasma is anticipated to be present as conjugated metabolites, an enzyme hydrolysis procedure has also been optimized as a component of the LC/MS/MS assay for elucidating the pharmacokinetic disposition of the parent markers and their metabolites.

Plasma concentrations of each of the parent chemical markers are measured simultaneously for determination of the pharmacokinetics of unchanged OMN54 components in human subjects. The corresponding plasma concentrations of conjugated OMN54 chemical markers are also determined for characterization of the pharmacokinetics of OMN54 conjugated metabolites.

Pharmacokinetic parameters including the area under the plasma concentration time course (AUC), total body clearance (CL), maximal plasma concentrations (Cmax), time to reach maximal plasma concentrations (Tmax), and apparent plasma elimination half-life (t½) are determined for each of the OMN54 chemical markers in plasma unchanged and as conjugated metabolites based on a non-compartmental approach. Plasma AUC data of unchanged OMN54 chemical markers and their metabolites will provide valuable information for an assessment of the systemic exposure of OMN54 in human subjects during the initial single dose-escalation cycle and during later repeated-dose administration cycles. Dose-linearity of plasma AUC data are evaluated for the presence of non-linear pharmacokinetics of OMN54 as an indication of potential saturable processes in the disposition of OMN54 in human subjects. Plasma half-lives of the OMN54 markers will also be evaluated following single and repeated-dose administration to evaluate for potential metabolic induction or inhibition interaction in the elimination of OMN54 upon chronic administration.

### Safety Assessments (Phase 1 Clinical Trials)

Safety criteria comprising the analysis include adverse events, serious adverse events, changes in clinical lab values from Screening, i.e., haematology, chemistry, and coagulation (INR and PTT), urinalysis, urine pregnancy tests, 12-lead ECG findings, vital signs, and physical exam changes from Screening, body weight, and review of events. Safety assessments monitored and recorded for the evaluation of tolerability and toxicity include:

Clinical labs—haematology, coagulation studies, i.e., INR and PTT, serum chemistry and urinalysis. Urine pregnancy testing for females of child-bearing potential can be performed.

Adverse Events graded according to the Cancer Therapy Evaluation Program—Common Terminology Criteria for Adverse Events (CTCAE, version 4.03).

Physical Examination findings

ECOG Performance Status

Vital signs (Temperature, Pulse, Respiration, and Blood Pressure)

12-Lead ECG

Body Weight (kg)

Toxicity was graded using the Common Terminology Criteria for Adverse Events (CTCAE) v4.03. A DLT (dose-limiting toxicity) is a toxicity that is considered as possibly, probably or definitely related to treatment with Aneustat™ (OMN54) and that is:

1. Hæmatological
Any CTCAE Grade 4 toxicity (CTCAE Grade 4 neutropenia has to be present for >7 days to constitute a DLT)
CTCAE Grade 3 or 4 neutropenia with fever
CTCAE Grade 3 or 4 thrombocytopenia associated with bleeding (excluding patients receiving therapeutic systemic anti-coagulation)

2. Non-Haematological

Any CTCAE Grade 3 or 4 toxicity despite adequate supportive care

CTCAE Grade 2 vomiting on 2 consecutive days despite optimal anti-emetic therapy

Any CTCAE Grade 2 toxicity that lasts longer than 2 weeks

3. Interruption of dosing for >2 weeks if that interruption is secondary to drug-related toxicity.

A Phase 1, open-label, multiple dose study was conducted to assess the safety, tolerability and pharmacokinetics of Aneustat™ (OMN54) administered on a daily oral regimen in patients with advanced cancer and lymphomas.

The primary (safety) objectives of the study were: (a) assessment of safety and tolerability of Aneustat™ (OMN54) in patients with advanced cancer and lymphomas; (b) determination of maximum tolerated dose (MTD) of two dosing regimens (once daily [QD] and twice daily [BID]) of Aneustat™ (OMN54); (c) determination of dose limiting toxicity (DLT) of two dosing regimens (once daily [QD] and twice daily [BID]) of Aneustat™ (OMN54); and (d) evaluation of the pharmacokinetic profile of Aneustat™ (OMN54) in cancer patients.

22 cancer patients were tested for 1,451 total days of dosing. The trials showed excellent safety profile of the Aneustat™ (OMN54) formulation.

A total of 225 adverse events (AE) of the following types were noted:

Gastrointestinal (GI) 64 (28%)—e.g., nausea, vomiting, abdominal pain

General 33 (15%)—e.g., edema, fatigue

Metabolic/Nutritional 29 (13%)—e.g., anorexia;

Investigations 25 (11%)—e.g., increased AST, ALP, weight loss . . .

Severity of the adverse events was determined according to the Severity Grading per US NCI Common Terminology Criteria for Adverse Events (CTCAE) v4.0 and graded as follows:

99 (44%) grade 1 (mild)

87 (39%) grade 2 (moderate)

35 (16%) grade 3 (severe)

4 (<2%) grade 4 (life threatening)

Four deaths, none of which were treatment related, occurred.

The attribution of the adverse events to treatment with Aneustat™ (OMN54) were categorized as follows:

188 (84%) not treatment-related

29 (13%) unlikely to be treatment-related

7 (3%) possibly treatment-related

1 (<0.05%) probably treatment-related

None were definitely treatment related

The 7 (3%) “possibly” treatment-related adverse events were noted as: nausea (mild); gastroesophageal reflux (moderate); gastroesophageal reflux (mild); dry, cracked hands (mild); vomiting (mild); bloating (mild); and constipation (mild).

The 1 (0.05%) “probably” treatment-related AE was vomiting (mild).

Amongst the 8 probably or possibly treatment related AEs, all but one were grade 1, mild severity.

### Assessment of Anti-Tumor Activity

Assessment of anti-tumour activity using standard response evaluation shows that OMN54 is effective under criteria (RECIST criteria, version 1.1; Ref. 13) and regulates one or more serological tumor markers, CA-125 (ovarian cancer and other cancers), CEA (colon cancer or lung cancer), PSA (prostate cancer), AFP (liver, testicular or ovarian cancer), CA 15.3 (breast cancer).

Appropriate radiological imaging tests and/or clinical measurements of the relevant body lesions are performed during administration and can be repeated approximately every 8 weeks. If there is evidence of CR or PR as per the RECIST criteria, version 1.1, the appropriate radiological scans and/or clinical measurements are repeated approximately 28-35 days after the initial observation for confirmation of the response. The RECIST criteria is used to assess objective response rate and stable disease, absence of disease or early progression.

### Biomarker Assays

Protein biomarkers in plasma samples are regulated by Aneustat™ (OMN54): C-reactive protein (CRP), IL-1b, IL-6, IL-8, IL-10, IL-12, IL-2ra, TNFα, IFN-gamma, VEGF, FGF, and GM-CSF. Assays are performed at the end of each cycle, typically 28 (+/−2) days to evaluate changes from the pre-dosing or pre-cycle initiation levels.

Phase IIA clinical studies to assess secondary objectives (efficacy and pathway biomarker studies) were conducted.

Preliminary assessment was made of of anti-tumor activity using standard response evaluation criteria (imaging) and tumor markers, as applicable, e.g., CA-125 (ovarian and others), CEA (colon and others), PSA (prostate), AFP (liver, testicular or ovarian), CA 15.3 (breast).

Evaluation of pathway biomarkers: C-reactive protein, IL-1b, IL-6, IL-8, IL-10, IL-12, IL-2ra, TNFα, IFN-gamma, VEGF, FGF, and GM-CSF were conducted to help characterize Aneustat™ (OMN54) activity.

### Therapeutic Activities

These compositions exhibit synergistic action with the therapeutic agent based on their anti-inflammatory, antioxidant, immune modulating, antiviral, antibacterial, antiproliferative activity or any combination of activities thereof.

The compositions demonstrate antioxidant activity which prevents damage to chromosomes/genes, reduces effect of mutagens, alleviates side-effects of chemotherapeutic agents, alleviates side-effects of hormone therapeutic agents, and enhances cell repair mechanisms.

The compositions further demonstrate immune system boosting activity which facilitates elimination of (i) damaged cells or (ii) cells with damaged genes. Further, the compositions provide general benefits of improving immune condition (passive immunotherapy).

The botanical sources of the extracts are botanicals that are essentially nontoxic with a long history of usage of the individual compounds/extracts. Anti-mutagenic properties as evidenced by Ames test results (together with increased sensitivity by synergism) reduce levels of chemotherapeutic agents necessary for treatment resulting in reduced toxicity for patients.

The botanical compositions demonstrate the ability to enhanced cell cycling which could make the botanical composition of the invention a powerful adjuvant to chemotherapy (e.g., with docetaxel), hormonal therapy, or radiation therapy by increasing effectiveness and reducing necessary dosages of chemotherapeutic agents and hormone therapeutic agents.

Quality control. IC₅₀ based compositions can be standardized based on specific activities of defined properties.

### EXAMPLES

Without further elaboration, it is believed that one skilled in the art can, using the preceding description, utilize the present invention to its fullest extent. The following examples are illustrative only, and not limiting of the remainder of the disclosure in any way whatsoever.

The following combinations of extracts were used throughout the examples: Ganoderma lucidum, Scutellaria barbata, optionally Scutellaria baicalensis and Salvia miltiorrhiza are the components of Aneustat™ also referred to as OMN54 in the specification. Aneutox comprises the same components in the same or different concentrations and additionally comprises, optionally, a chemotherapeutic agent.

In addition, the compositions of the invention may include, optionally, Panax Quinquefolium (Western ginseng), Camellia sinensis (green tea), and Hippophae rhamnoides (sea buckthorn). Results obtained with these combinations or the individual extracts were often compared with ACAPHA, a combination of six herbs (Sophora tonkinensis, Polygonum bistorta, Prunella vulgaris, Sonchus brachyotus, Dictamnus dasycarpus and Dioscorea bulbifera).

One skilled in the art would appreciate that while the foregoing examples are illustrative of the invention, any cell line may be used. For example, although not limiting, cells may be obtained from ATCC, Rockville, Md.

One skilled in the art would also appreciate that while the foregoing examples are illustrative of the invention, multiple prostate cancer in vivo models may be used. For example, CaP xenografts in mice may be utilized. Additionally, a Pten knockout mouse strain, in which heterozygous mice develop tumors of the uterus, prostate, thyroid, colon, and adrenal medulla, may be obtained from the Mouse Models of Human Cancers Consortium (Podsypanina K. et al., (1999) Proc. Nat'l Acad. Sci. USA 96: 1563-1568).

### Example 1

### Methods for Preparation of Botanical Extracts

The compositions of the present invention may be administered as dried herbs. Botanical preparations contain phytochemicals some of which are soluble in aqueous media while others are relatively more soluble in organic (alcohol, lipid) media. Different extraction methods were used and tested for the ability to extract effective ingredients from the herbs. Extraction methods include: aqueous (hot water) extraction; organic (lipid fraction) extraction; non-alcohol organic (e.g., ethyl acetate, ester) extraction; and alcohol (e.g., ethanol) Extraction.

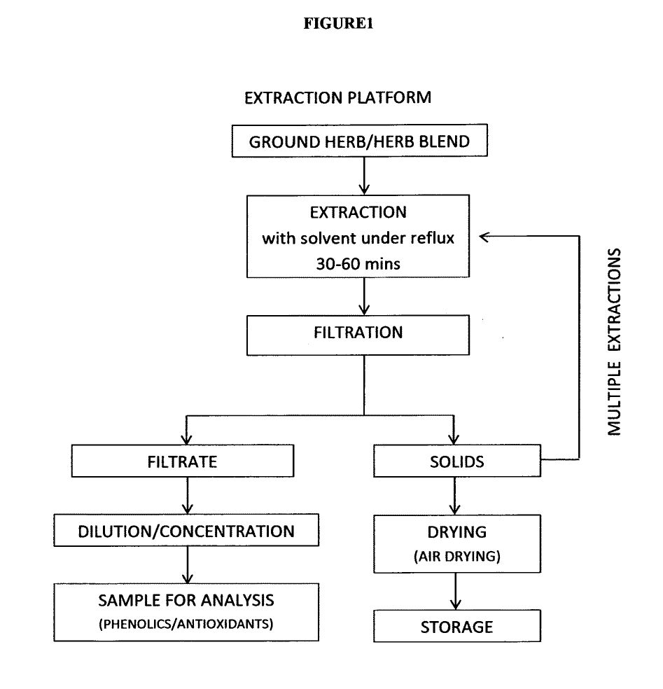

Products are prepared from herbs using different solvents by the general extraction platform shown in FIG. 1. Herb or herb blends were extracted with solvent (hot water, 80% ethanol, or ethyl acetate) under reflux for 30-60 minutes, separated by filtration to obtain a filtrate, and air dried for further analysis. The filtrates were combined, diluted or concentrated prior to determination of activities.

In one embodiment, HPLC grade ethyl acetate was used as a solvent at ratios of the solvent to the herb ranging from 5.0:1, 5.8:1, 6.0:1, 7.0:1, 8.0:1, 9.0:1, 10:1, etc. In a typical procedure solvent to raw material ratio is at least 6.1:1 for Ganoderma lucidum, at least 5.8:1 for Salvia miltiorrhiza, and at least 8.0:1 for Scutellaria barbata.

Extraction is carried out at initially at 90-100° C. and then with gentle simmering at 80-85° C. for 30-60 mins. The extract is then mixed and cooled. To be acceptable, total solid content of the extract after extraction but before first concentration/evaporation step should be at least 0.3% for Ganoderma lucidum, at least 0.05% for Salvia miltiorrhiza, and at least 0.3% for Scutellaria barbata. A first and optionally a second, stage of concentration is then carried out using a rotary evaporator. In a preferred embodiment, total solid content of the extract after extraction but before first concentration/evaporation is at least 5% for Ganoderma lucidum, 1% for Salvia miltiorrhiza, and 5% for Scutellaria barbata.

The solids left behind after the extraction step may be re-extracted and the entire process repeated. Ranges of typical yields from a batch are expected to be 2-3% for Ganoderma lucidum, 0.3-1% for Salvia miltiorrhiza, and 2-3% for Scutellaria barbata.

### Example 2

### Maximum Tolerable Dose of Formulations of the Invention

A solution/suspension of Ganoderma lucidum, Salvia miltiorrhiza, and Scutellaria barbata representing 10× IC₅₀ was administered to immune compromised mice (Rag2m, 10 female) and also to immune competent mice (CDI, 5 male, 5 female). A solution of the extracted material (43.65 mg/ml) was administered orally (1 ml/day/animal) to SCID/nod mice (25 gm; n=5) once a day for up to 14 days. The mice were monitored over a 28-day period for signs of stress following drug administration, including substantial loss of body weight, diarrhea, heavy panting, ruffling of hair, etc. On days 2 through 14, less than 13% body weight loss was observed (FIG. 11) and the animals were considered to be healthy. At the end of the period mice were terminated by CO2 inhalation. Age-matched control mice (n=4) were treated with saline 1 ml/day for the 14 days. The data shows the single maximum tolerated dose for two formulations with and without emulsifying agents:

Formulation 1 (without emulsifiers):

| | |
|---|---|
| OMN54 drug substance | 100 mg |
| Soya Oil, USP | 160 mg |
| Alpha tocopherol (Vit E), USP | 1 IU |

The Single maximum Tolerated Oral Dose (MTD) for Formulation 1 was 3.4 g/kg for immune compromised mice and 4.1 g/kg for immune competent mice.

Formulation 2 (with emulsifiers):

| | | |
|---|---|---|
| OMN54 drug substance | API | 100 mg |
| Ascorbic Acid 6-Palmitate | Antioxidant Preservative | 1.1 mg |
| Cremophor EL | Emulsifying Agent | 17.4 mg |
| Emersol 6313 Oleic Acid | Emulsifying Agent | 8.8 mg |
| Labrasol | Emulsifying Agent | 17.4 mg |
| Soya Oil, USP | Diluent | 72.6 mg |
| Alpha tocopherol (Vit E), USP | Antioxidant Preservative | 1.1 mg |
| (1.1 IU/g) | | |

The Single maximum Tolerated Oral Dose (MTD) for Formulation 1 was 8.652 g/kg for immune compromised (ICR) mice.

Formulation 2 of OMN54 did not cause significant changes in ICR mice, such as abnormal ingestion, drinking, stool, urine, behavior, activity, body weight and death.

### REFERENCES

1. Saunders P R. Reishi. In: Herbs Everyday Reference for Health Professionals, Ed. F. Chandler. Canadian Pharmacists Association and Canadian Medical Association 2000 p 181-184.
2. Reishi Mushroom Ganoderma lucidum. American Herbal Pharmacopoeia and Therapeutic Compendium. 2000.
3. Bensky D, Gamble A. Dan Shen. In:Chinese Herbal Medicine Materia Medica, Eastland Press 1993 p. 267-268.
4. Bensky D, Gamble A. Ban Zhi Lian. In: Chinese Herbal Medicine Materia Medica, Eastland Press 1993. p 102.
5. McGuffin M, Kartesz, J T, Leung A Y, Tucker A O. Herbs of Commerce. Second edition. American Herbal Products Association. 2000.
6. Ganoderma. In: Pharmacopoeia of the People's Republic of China (English Edition 2000) Vol 1, Chemical Industry Press. p. 93
7. Chang R., 1994. “Effective Dose Of Ganoderma In Humans.” Proceedings of Contributed Symposium 59 A,B. 5th International Mycological Congress. Vancouver. pp. 117-121.
8. Radix Salviae Miltiorrhizae. In: Pharmacopoeia of the People's Republic of China (English Edition 2000) Vol 1, Chemical Industry Press. p. 192-193
9. Zhou L, Zuo Z, Chow M S. Danshen: an overview of its chemistry, pharmacology, pharmacokinetics, and clinical studies. J Clin Pharmacol 45(12):1345-1359, 2005.
10. Herba Scutellariae Barbatae. In: Pharmacopoeia of the People's Republic of China (English Edition 2000) Vol 1, Chemical Industry Press. p. 118
11. Chang M Y. Anticancer Medicinal Herbs, Hunan Science and Technology Publishing House, Changsha PRC. 1992 pp 90-93.
12. Dharmananda S. Oldenlandia and Scutellaria—Antioxidant and Anticancer Herbs. www.itmonline.org/art/oldenlandia.htm.
13. Eisenhauer, E. A, Therasse, P., Bogaerts, J., Schwartz, L. H., Sargent, D., Ford, R., Dancey, J., Arbuck, S., Gwyther, S., Mooney, M., Rubenstein, L., Shankar, L., Dodd, L., Kaplan, R., Lacombe, D. and Verwij, J.: New Response Evaluation Criteria in Solid Tumors: Revised RECIST Guideline (version 1.1). European Journal of Cancer 45(2009) 228-247.

All publications and patent applications cited in this specification are herein incorporated by reference as if each individual publication or patent application was specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A formulation comprising:
two or more of an extract of Ganoderma lucidum, an extract of Salvia miltiorrhiza, an extract of Scutellaria barbata, and an extract of Scutellaria baicalensis wherein each extract comprises about 1 to about 90 percent by weight; and
at least one emulsifying agent.

2. The formulation of claim 1 wherein the emulsifying agent is selected from a fatty acid, oleic acid, polyoxyethylene glycerol ester of fatty acids, Tagats, polooxylated castor oil, ethylene glycol esters, glycol stearate, glycol distearate, propylene glycol ester, propylene glycol myristate; glyceryl ester of fatty acids, glyceryl stearates, glyceryl monostearate, sorbitan ester, Span, Tween; polyglyceryl esters, polyglyceryl 4-oleate, fatty alcohol ethoxylates, Brij type emulsifiers, ethoxylated propoxylated block copolymers, poloxamers, polyethylene glycol esters of fatty acids, Labrafils, Labrafacs, and Labrasols, cremophores, glycerol monocaprylate/caprate, Campmul CM 10, Gelucire, Capryol, Captex, Acconon, transcutol, and triacetin.

3. The formulation of claim 1 wherein the emulsifying agent is selected from Cremophor EL, oleic acid and labrasol.

4. The formulation of claim 1 further comprising an antioxidant.

5. The formulation of claim 4 wherein the antioxidant is selected from ascorbic acid and alpha tocopherol.

6. The formulation of claim 1 further comprising a diluent.

7. The formulation of claim 6 wherein the diluent is soya oil.

8. The formulation of claim 1 wherein the extract is made in a non-alcoholic organic solvent.

9. The formulation of claim 8 wherein the non-alcoholic organic solvent is an ester.

10. The formulation of claim 9 wherein the ester is ethyl acetate.

11. The formulation according to any of claims 1-10, wherein the formulation exhibits increased bioavailability compared to a formulation without an emulsifying agent.

12. The formulation according to any of claims 1-10, wherein the formulation exhibits increased maximum tolerated dose (MTD) compared to a formulation without an emulsifying agent.

13. The formulation according to claim 1, comprising:
an ethyl acetate extract of Ganoderma lucidum, an ethyl acetate extract of Salvia miltiorrhiza, and an ethyl acetate extract of Scutellaria barbata wherein each extract comprises about 1 to about 90 percent by weight;
at least one emulsifying agent selected from Cremophor EL, oleic acid and labrasol;
an antioxidant selected from ascorbic acid and alpha tocopherol; and
a diluent selected from soya oil.

14. The formulation according to any of claims 1 through 13, further comprising a chemotherapeutic agent.

15. The formulation of claim 14, wherein the chemotherapeutic agent is selected from carboplatin, Navelbine® (vinorelbine), anthracycline (Doxil), lapatinib (GW57016), Herceptin®, gemcitabine (Gemzar®), capecitabine (Xeloda®), Alimta®, cisplatin, 5-fluorouracil, epirubicin, cyclophosphamide, Avastin®, Velcade®, paclitaxel and docetaxel.

16. The formulation of claim 14, wherein the chemotherapeutic agent is selected from the group consisting of antimetabolites, nucleoside analogs, platinum-based agents, alkylating agents, tyrosine kinase inhibitors, anthracycline antibiotics, vinca alkloids, proteasome inhibitors, macrolides, and topoisomerase inhibitors.

17. A method for treating a subject suspected of having cancer or a related disease, the method comprising: administering an amount of the formulation according to any of claims 1-15, sufficient for alleviating a symptom of cancer or a related disease.

18. The method of claim 17, wherein the formulation comprises 0.1 to 100 mg of an active ingredient consisting of two or more of an extract of Ganoderma lucidum, an extract of Salvia miltiorrhiza, an extract of Scutellaria barbata, and an extract of Scutellaria baicalensis.

19. The method of claim 18 wherein 1, 1.5, 2. 2.5, 3, 4, or 5 g of the active ingredient is administered daily.

20. The method of claim 18, wherein the formulation is administered in 7, 14 or 28 day cycles of 1, 2, 3, 4, 5, or 6 cycles.

21. The method of claim 17, further comprising administering a chemotherapeutic agent either in the same formulation or separately administered as part of a therapeutic regimen.

22. The method of claim 21, wherein the chemotherapeutic agent is selected from carboplatin, Navelbine® (vinorelbine), anthracycline (Doxil), lapatinib (GW57016), Herceptin®, gemcitabine (Gemzar®), capecitabine (Xeloda®), Alimta®, cisplatin, 5-fluorouracil, epirubicin, cyclophosphamide, Avastin®, Velcade®, paclitaxel and docetaxel.

23. The method of claim 21, wherein the chemotherapeutic agent is selected from the group consisting of antimetabolites, nucleoside analogs, platinum-based agents, alkylating agents, tyrosine kinase inhibitors, anthracycline antibiotics, vinca alkloids, proteasome inhibitors, macrolides, and topoisomerase inhibitors.
